(19) United States
(12) Patent Application Publication
(10) Pub. No.: US 2008/0082114 A1
(43) Pub. Date: Apr. 3, 2008

(54) Adhesive Mechanical Fastener for Lumen Creation Utilizing Tissue Necrosing Means

(76) Inventors: Robert H. McKenna, Bountiful, UT (US); Frederick E. Shelton, Hillsboro, OH (US); Mark S. Ortiz, Milford, OH (US); James W. Voegele, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 11/536,741

(22) Filed: Sep. 29, 2006

(51) Int. Cl. A61B 17/08

(52) U.S. Cl. 606/153

(57) ABSTRACT

A two piece anastomosis device for attaching two organs together and creating a passage between the organs is disclosed. The anastomosis device has a first tissue clamping ring and a second tissue clamping ring that are brought together to clamp tissue therebetween and cut off the flow of blood to the tissue. The tissue clamping rings are locked together with an adhesive, and over time, causes the clamped tissue to necrose and slough off. The sloughed tissue creates a passageway through the anastomosis device. A method of using the fastener to create a bypass passageway between the stomach and small intestine is disclosed.

### FIELD OF THE INVENTION

The present invention relates, in general, to surgical devices for the anastomosis of organs, and in particular to tissue fasteners, surgical devices for deploying tissue fasteners, and methods of use during surgery.

### BACKGROUND OF THE INVENTION

Obese patients can have increased morbidity from excess weight. The extra weight can place strain on body circulatory systems, resperatory systems, the digestive system, and can stress or overload body organs. This can cause numerous medical conditions such as diabetes, high blood pressure, high cholesterol, sleep apnia, and can increase the risk of a major coronary event. Bariatric medicine is focussed on the prevention, control, and treatment of obesity. Diet, drugs, and surgery are the most common tools used to reduce a patient's calloric intake, reduce their weight, and save their lives.

Bariatric surgical techniques can result in dramatic weight loss for the morbidly obese, and can add years to a patients life. These surgeries can include stomach stapling to create a small pouch, intestinal bypass surgery, gastric banding, placement of a large filler object in the stomach to reduce the internal volume of stomach, and gastric sleeves.

The small intestine is attached to the pyloric valve at the base of the stomach. The pyloric valve regulates the passage of partally digested food from the stomach and into the first portion of the small intestine. The first foot and a half to two feet of the small intestine absorbs the majority of the nutrients from the partially digested food as it passes through. Intestinal bypass surgery is focussed on bypassing this section of the intestine with surgical cutting and stapling procedures such as a Roux-en-Y.

Roux-en-Y is a surgical stapling procedure that severs the small intestine below the initial two foot segament of nutrinet absorbing tissue. The long section of the severed intestine is brought up and anastomosed or attached to another organ such as the side of the stomach and an opening is cut in the side of the stomach at the anastomosis point to allow food to exit from the stomach and into the new section of intestine. The short section of intestine extending from the pyloric valve of the stomach is attached to the side of the long section of severed intestine below the stomach anastomosis point and a second opening is cut between the two portions of intestine.

The foregoing examples are merely illustrative and not exhaustive. While a variety of techniques and devices have been used in bariatric surgery, no one has previously made or used an anastomosis instrument in accordance with the present invention. Other aspects of the present teaching relate to novel anastomosis fasteners and instruments that are not limited to intestinal bypass surgery.

Consequently, a significant need exists for an improved anastomosis fastener and device to deploy the fastener that is easy to use, low in cost, provides increased security and device and can create an anastomosis between body organs, and can be used for bariatric surgery.

### BRIEF SUMMARY OF THE INVENTION

In accordance with the present invention, there is provided an anastomosis device for attaching two hollow organs together and creating a passage therebetween. The anastomotic device includes a first tissue clamping ring, a second tissue clamping ring, and an adhesive for locking the first tissue clamping ring and the second tissue clamping ring together. The first tissue clamping ring and the second tissue clamping ring are locked in a spaced apart orientation to clamp tissue between the first tissue clamping ring and the second tissue clamping ring.

Also in accordance with the present invention, there is provided a method of creating a passage in tissue between a pair of hollow organs. First, the method includes a step of providing an anastomotic device for clamping on tissue. The anastomotic device includes a first tissue clamping ring, a second tissue clamping ring, and an adhesive in at least one of the first tissue clamping ring and the second tissue clamping ring. Second, the step of aligning a first hollow organ with a with a second hollow organ in a desired orientation is performed. Third, the step of placing the first tissue clamping ring or the second tissue clamping rings into the first hollow organ is performed. And, placing the other of the first tissue clamping ring or second tissue clamping ring in opposition in the second hollow organ. Fourth, the first tissue clamping ring and the second tissue clamping rings are clamped together in a spaced apart orientation to clamp tissue therebetween, and lock the first and second rings together with the adhesive.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is an isometric view of a two piece fastener and a surgical instrument that can deploy a two part fastener to create a lumen between two body structures of a patient.

FIG. 2 is an isometric view of the two piece fastener of FIG. 1.

FIG. 3 is a cross sectional view of the two piece fastener of FIG. 2 showing sealed ampoules of adhesive therein.

FIG. 4 is a partial cross sectional view of a patient's stomach and an upper portion of the small intestine showing a dashed line placement of the two piece fastener to create a lumen between the stomach and small intestine.

FIG. 5 is a partial cross sectional view of the stomach and an upper portion of the small intestine of FIG. 4 and showing the first steps of a bariatric surgical method to create a lumen between the stomach and small intestine.

FIG. 6 is a partial side cross sectional view showing a trocar of the surgical device penetrating through the small intestine and stomach, and a grasper moving a second clamping member with attached second clamping ring thereon, onto the trocar.

FIG. 7 is a partial side cross sectional view of FIG. 6 showing the distal clamp member being held by the grasper after attachment onto to the trocar of the fastener applying instrument.

FIG. 8 is a partial side cross sectional view of FIG. 7 showing the second clamp ring closed to a firing position, and the first clamp ring moved to the fired position to close the two part fastener onto tissue and lock the two part fastener together with an adhesive.

FIG. 9 is a partial side cross sectional view of FIG. 8 showing the two piece fastener adhered together in tissue, with the fastener applying instrument and distal clamp member being removed.

FIG. 10 is a partial side cross sectional view of FIG. 9 showing the two piece fastener adhered together to pinch tissue therebetween, and with an incision in the intestine closed.

FIG. 11 is a is a partial side cross sectional view of FIG. 10 showing the two piece fasstener glued together and the tissue pinched therebetween necrosed from blood loss and being sloughed off into the intestine to create an open passage between the stomach and small intestine through the two part fastener.

FIG. 12 is an isometric view of an alternate two piece fastener.

### DETAILED DESCRIPTION OF THE INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

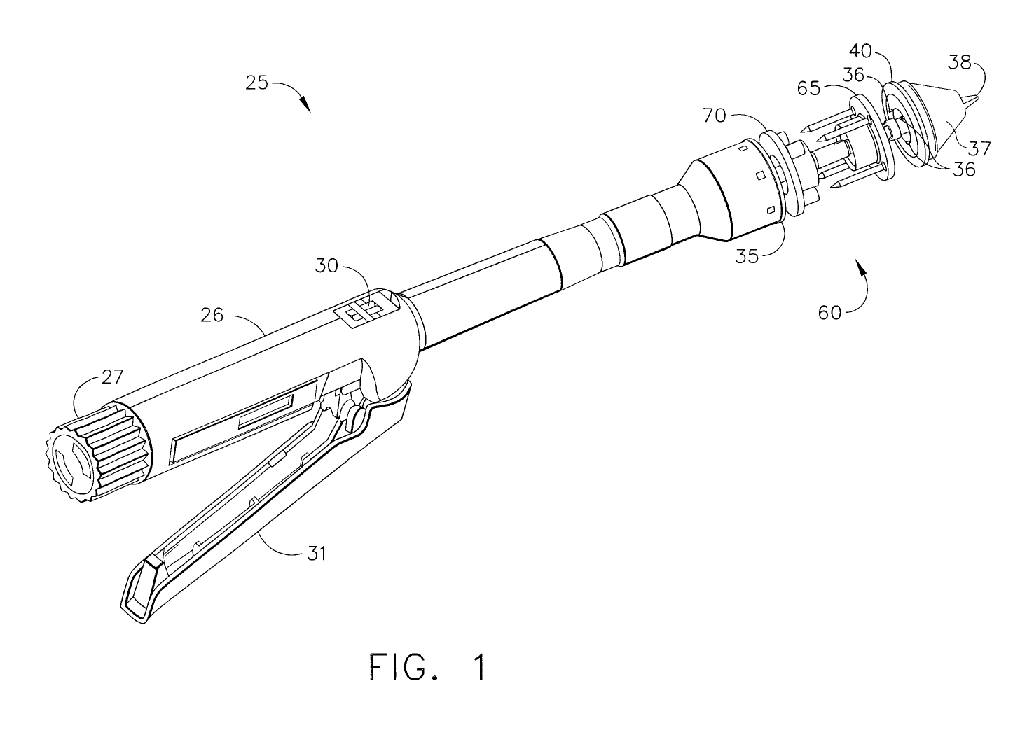

Turning to the Figures, wherein like numerals denote like components throughout the several views, in FIG. 1, a two-piece anastomosis fastener 60 and an anastomosis fastener applying instrument 25 that can be used to join two organs or organ sections together are shown. Fastener 60 is releasably attached to fastener applying instrument 25 but is shown spaced away from the attachment points for illustrational purposes. Fastener 60 has a first clamping ring 65, and a second clamping ring 70 and when used on a patient, first clamping ring 65 and second clamping ring 70 fasten together to clamp and hold organs in juxtaposition, and over time, create an open passage through the organs. First clamping ring 65 and second clamping ring 70 lock together with an adhesive.

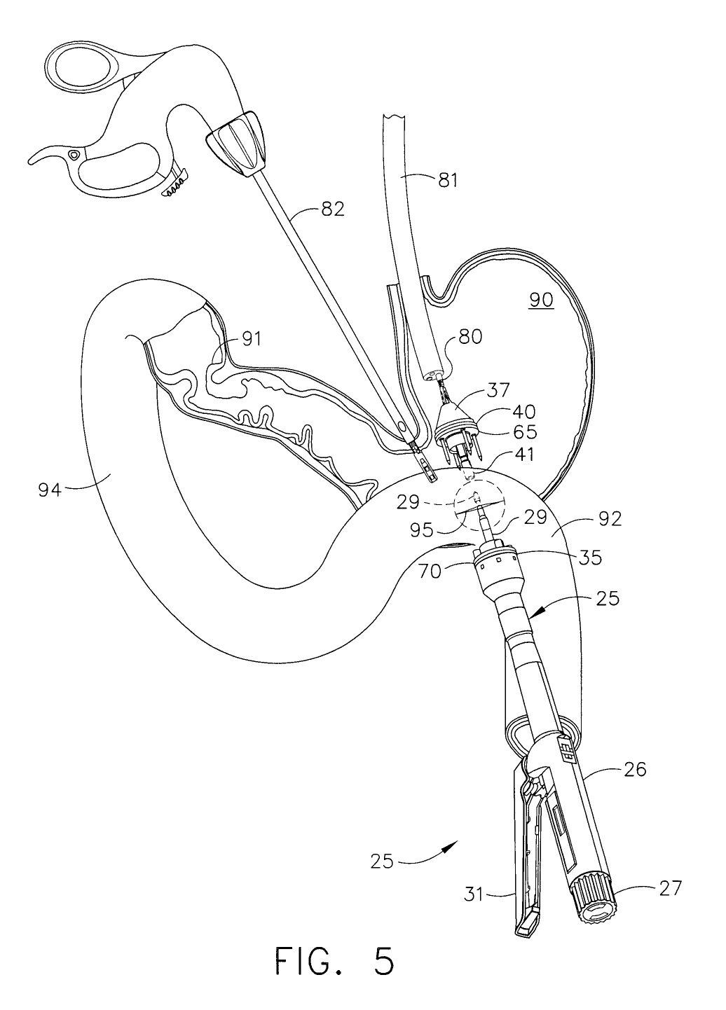

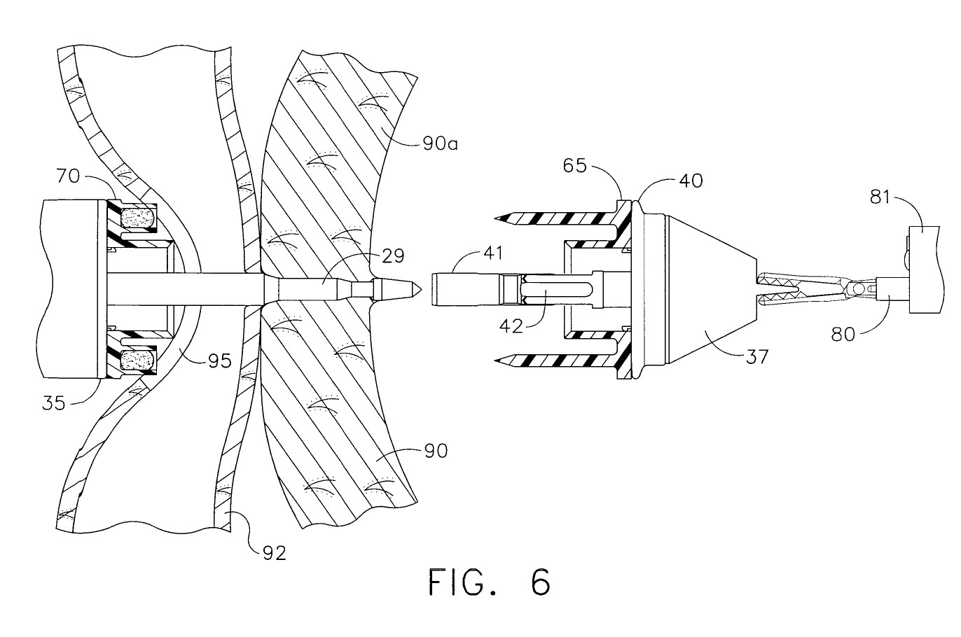

Fastener applying instrument 25 has a proximal clamp member 35 and a distal clamp member 40, both of which are moveable longitudinally. Distal clamp member 40 is moveable from an open position to a closed or fired position, and is releasably removable from a trocar 29 (FIGS. 5 and 6) when fastener applying instrument 25 is in a full open position of in FIG. 1. Proximal movement of distal clamp member 40 towards proximal clamp member 35 brings first clamping ring 65 and second clamping ring 70 of fastener 60 partially together to a firing position. The action of moving distal clamp member 40 towards the firing position locks distal clamp member 40 onto trocar 29 of the fastener applying instrument 25, and moves the distal clamp member 40 proximally as described below. When distal clamp member 40 is at a proximal most firing position, the proximal clamp member 45 can be unlocked and fired to clamp and lock first clamping ring 65 and second clamping ring 70 together to create the anastomosis.

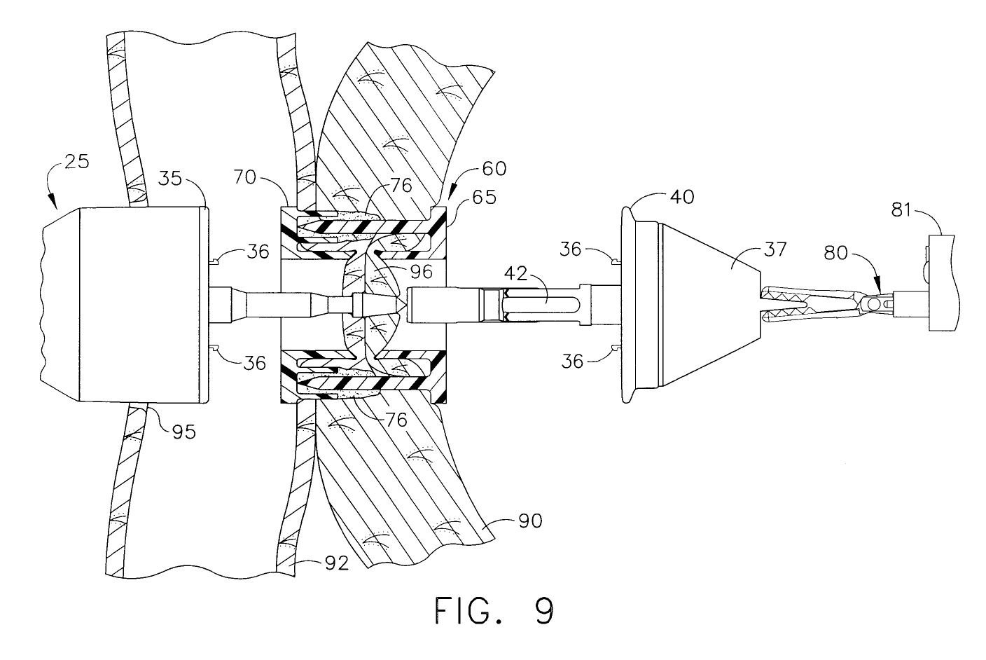

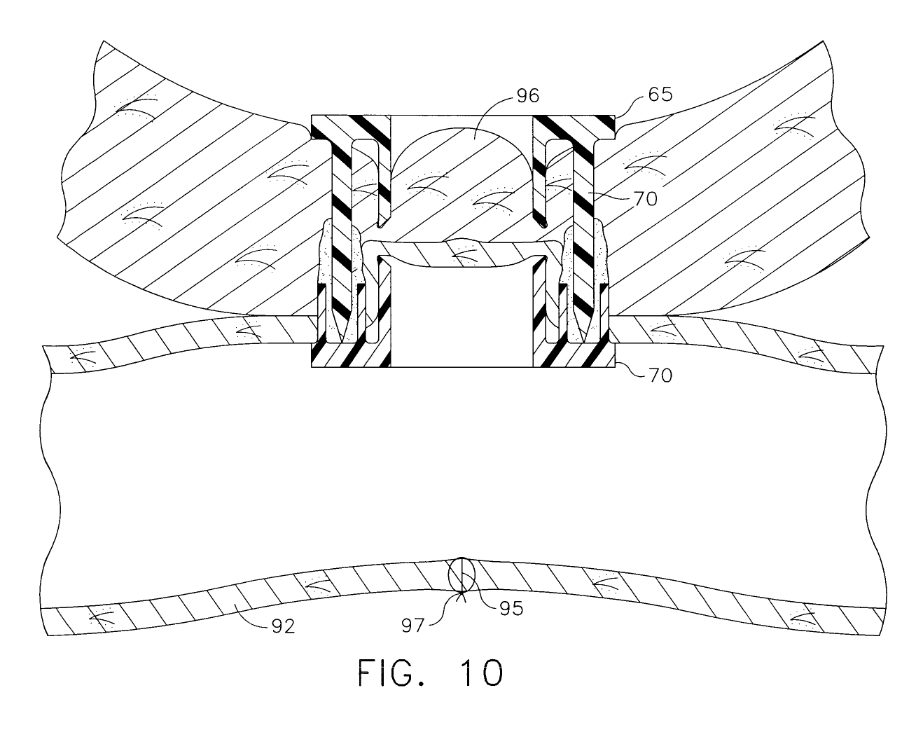

The fastener applying instrument 25 has a body 26, a knob 27, a firing trigger 31 and a gauge 30. Knob 27 is coupled to the distal member 40 by a rod 28 extending longitudinally within fastener applying instrument 25, and terminating in a distal pointed trocar 29. Knob 27 is threadably connected to rod 28 at a proximal end such that rotation of knob 27 moves rod 28 and trocar 29 (and distal clamp member 40 when attached) proximally or distally depending on rotational direction. As shown in FIG. 6, distal clamp member 40 has a proximally extending hollow shaft 41 with locking clamps 42 that can releasably attach to pointed trocar 29. Distal clamp member 40 is removable when fastener applying instrument 25 is in a full open position as shown in FIG. 1. Firing trigger 31 is operably coupled to proximal clamp member 35 such that actuation of firing trigger 31 moves proximal clamp member 35 distally when distal clamp member 40 is in the firing position to secure first clamping ring 65 and second clamping ring 70 of fastener 60 together to create the anastomosis. Gauge 30 could be provided to indicate to the surgeon when distal clamp member 35 is in the firing position and when proximal clamp member 35 is unlocked. By way of example, retention features 36 (see FIG. 9) such as snaps, clamps, grips, friction features, mild adhesives, tacky surfaces and the like may be located on proximal clamp member 35 and distal clamp member 40 to releasably retain first clamping ring 65 and second clamping ring 70 thereon. Retention features 36 can also align first clamping ring 65 and second clamping ring 70 relative to each other. An example of a fastening instrument that that has a moveable and removable distal clamping member, distally moving firing mechanisms, and gauge mechanisms is disclosed in U.S. Pat. No. 5,533,661 entitled “Sealing Means for Endoscopic Surgical Anastomosis Stapling Instrument” by L. Main et al , the teachings of which are hereby incorporated by reference in their entirety.

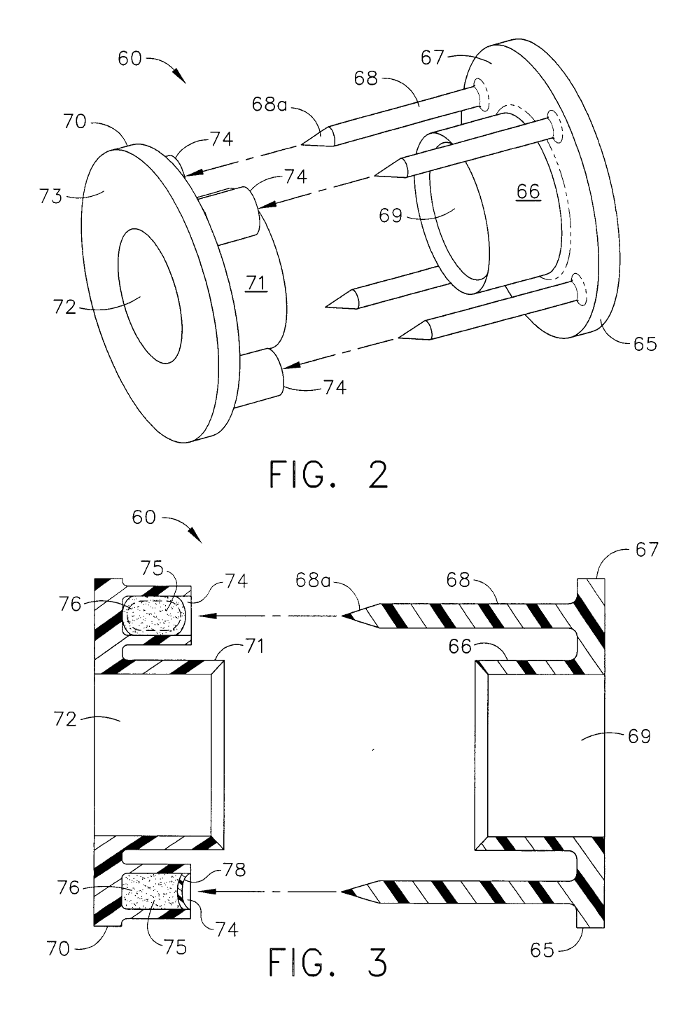

FIG. 2 is an isometric view of the example of the two-piece anastomosis fastener 60 of FIG. 1 with first clamping ring 65 and second clamping ring 70 separated and spaced apart. First clamping ring 65 has a first ring 66 defining a first passageway 69, and a first flange 67. Spacers 68 extend from first flange 67 about first ring 66 and have points 68a thereon. Second clamping ring 70 can have a second ring 71 defining a second passageway 72, and a second flange 73. Sockets 74 (FIG. 3) could be located about second ring 71 and may receive spacers 68 from the first clamping ring 65. Two-piece anastomosis fastener 60 can be made from a number of non- absorbable implantable materials such as but not limited to metallic materials such as stainless steel, gold, or titanium, and plastics such as but not limited to polyethylenes, polyetheretherketones (PEEK) and bioabsorbables such as polylactic acid (PLA), polyglycolic acid (PGA) and their copolymers. Alternately, two-piece anastomosis fastener 60 can be made from a number of absorbable implantable materials such as but not limited to as polylactic acid (PLA), polyglycolic acid (PGA) and their copolymers, and absorbable materials used for sutures such as dexon, vicryl, polydioxanone, and polyglyconate.

FIG. 3 is a cross sectional view of the example two-piece anastomosis fastener 60 showing passageways 69, 72 and sockets 74. Sealed containers, bladders, or ampoules 75 could be fixably located in sockets 74 and can contain an adhesive 76, an additive 77 such as an initiator 77. Adhesive 76 could be released from frangible ampoules 75 when penetrated or ruptured such as by points 68a of spacers 68 when first clamping ring 65 and second clamping ring 70 are clamped together. Ampoules 75 could be sealed to provide stability, increase shelf-life, and prevent setting of adhesive 76. Ampoules 75 could be made of any one of a number of materials or combinations of materials such as but not limited to coatings, glasses, plastic materials, metallic materials, gels, or ceramics. By way of example, plastic materials could include butyrate or polyethylene rubber, silicone or plastic material, such as, for example, polyvinyl chloride, polyethylene, polyurethane, natural or nitril rubber, or any combination thereof. Other materials, such as a pierceable metal as aluminum or gold may be used for the ampoule 75. An example of a suitable ampoule that can be used is disclosed in, for example, U.S. Pat. No. 5,928,611, the entire disclosure of which is incorporated herein by reference. Alternately, for example, another type of ampoule 75 could be made by placing adhesive 76 into the bore of the sockets 74 and inserting or applying a barrier material or plug 78 into the sockets 74 over the adhesive 76 to act as a seal or airtight seal for adhesive 76. Plug 78 could be any of the materials listed above for ampoules 76 or any other material that could act as a barrier.

By way of example, adhesive 76 could be a single part or a dual part adhesive that is a polymerizable and/or cross-linkable material such as but not limited to a cyanoacrylate adhesive. The adhesive material, for example, may be but not limited to a monomeric (including prepolymeric) adhesive composition, a polymeric adhesive composition, or any other natural or artificial compound that can adhere first clamping ring 65 and second clamping ring 70 together. In embodiments, the monomer may be a 1,1 -disubstituted ethylene monomer, e.g., an alpha-cyanoacrylate. When cross linked, the cyanoacrylate changes from a liquid to a solid. Cross linked adhesive 76a can be a rigid or flexible and can be non-permeable or permeable. If desired, adhesive 76 can be a single part or dual part adhesive, and/or can contain one or more additives 77. Examples of suitable additives 77 include, but are not limited to, anesthetics, sclerotic or necrosing agents plasticizing agents, thixotropic agents, buffers, catalysts, adhesive initiators, fillers, micro particles, thickeners, solvents, drugs, medicaments, natural or synthetic rubbers, stabilizers, pH modifiers, bioactive agents, cross-linking agents, chain transfer agents, fibrous reinforcements, colorants, preservatives, formaldehyde reducing or scavenging agents, flavorants, perfumes, mixtures thereof, and the like. Other suitable single part and dual part adhesive barrier materials 76 and additives 77 may be found in U.S. Application 20040190975 by Goodman et al. which is hereby incorporated by reference in its entirety. Additionally, by way of example, ampoule 75 may contain more than one separate chamber therein with adhesive in one chamber and an additive 77 in the other. Dual chamber ampoules could also be used for two part adhesives which would mix when penetrated, for polymerization initiators, or any drugs or medicaments, or any other additives listed above. Alternately, instead of adhesive 76, ampoules 76 could solely contain additives 77 such as adhesive initiators or compounds to “kick” or induce adhesive setting, or any other additives 77 listed above with or without adhesive 76. By way of example, ampoules 75 of adhesive 76 or additives 77 can be placed in any order in any socket 74 if desired and, more than one ampoule 75 could also be placed in a socket 74.

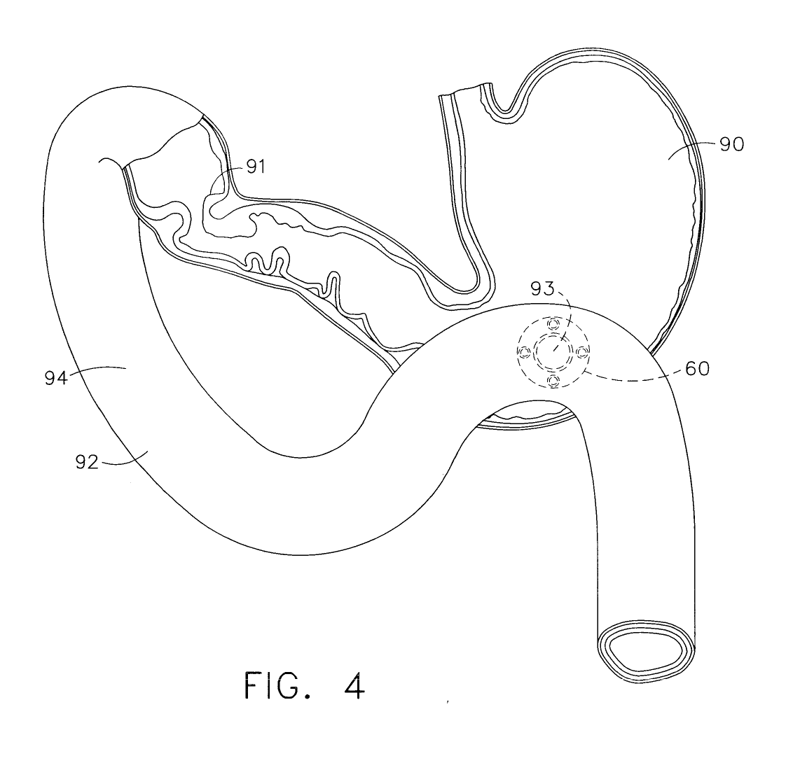

FIG. 4 is a partial cross sectional view of a stomach 90, a pyloric valve 91 and a small intestine 92 descending from the pyloric valve 91. Anastomosis fastener 60 (dashed lines) is shown placed within small intestine 92 and stomach 90 to create an open passageway 93 between stomach 90 and small intestine 92. The passageway 93 allows food to exit from the stomach 90 into the small intestine 92 and generally bypass the first foot and a half to two feet of the small intestine. This initial intestinal portion, or duodenum 94 of the small intestine absorbs the majority of the nutrients from food. The food bypassing through passageway 93 in anastomosis fastener 60 reduces the amount of food that can pass through the duodenum 94, and reduces the amount of nutrients or calories that can be absorbed by a patient. The unabsorbed nutrients that bypass through passageway 93 will pass through the patient and be expelled.

FIG. 5 shows the first steps in the gastric bypass surgery used to place anastomosis fastener 60 into a patient with the fastener applying instrument 25. The surgery depicted is minimally invasive and uses a combination of a natural body orifice and endoscopic ports to perform. Initially, the patient's abdomen is insufflated and an endoscope (not shown) is used to view the exterior of the stomach 90 and the duodenum 94. Outside of the patient, distal clamp member 40 with attached first clamping ring 65 is removed from fastener applying instrument 25. Hollow shaft 41 of distal clamp member 40 is grasped by a grasper 80 extending from a working channel of a second endoscope 81. The assembly of distal clamp member 40 and attached first clamping ring 65 are inserted into a patients stomach through the mouth and esophagus with a blunt atraumatic cone 37 of distal clamp member 40 leading. Once in the stomach, grasper 80 releases the hollow shaft 41 of distal clamp member 40, and distal clamp member 40 with attached first clamping ring 65 is released from grasper 80, reoriented 180 degrees, and re-grasped with grasper 80 as shown in FIG. 5.

A second grasper 82 is inserted into the abdomen and grasps and positions small intestine 92 as shown against a side of the stomach 90. An incision 95 is placed through one wall of small intestine 92 below the duodenum 94. Fastening applying instrument 25 is then inserted into the abdomen, with the body 26 outside the abdomen, and trocar 29 is guided into incision 95 in small intestine 92. If desired, an atraumatic sleeve or dummy cap can be placed over trocar 29 to blunt trocar 29 as fastener applying instrument 25 is inserted into the abdomen and into position (not shown). The sleeve or dummy cap can be removed from the trocar 29 prior to placement of trocar 29 to the position of FIG. 6.

As shown in FIG. 6, the pointed trocar 29 of fastener applying instrument is pushed through the uncut wall of small intestine 92 and partially through a wall 90a of stomach 90. Endoscope 81 and grasper 80 are being used to position and align hollow shaft 41 of distal clamp member 40 with trocar 29 protruding through the wall 90a of stomach 90.

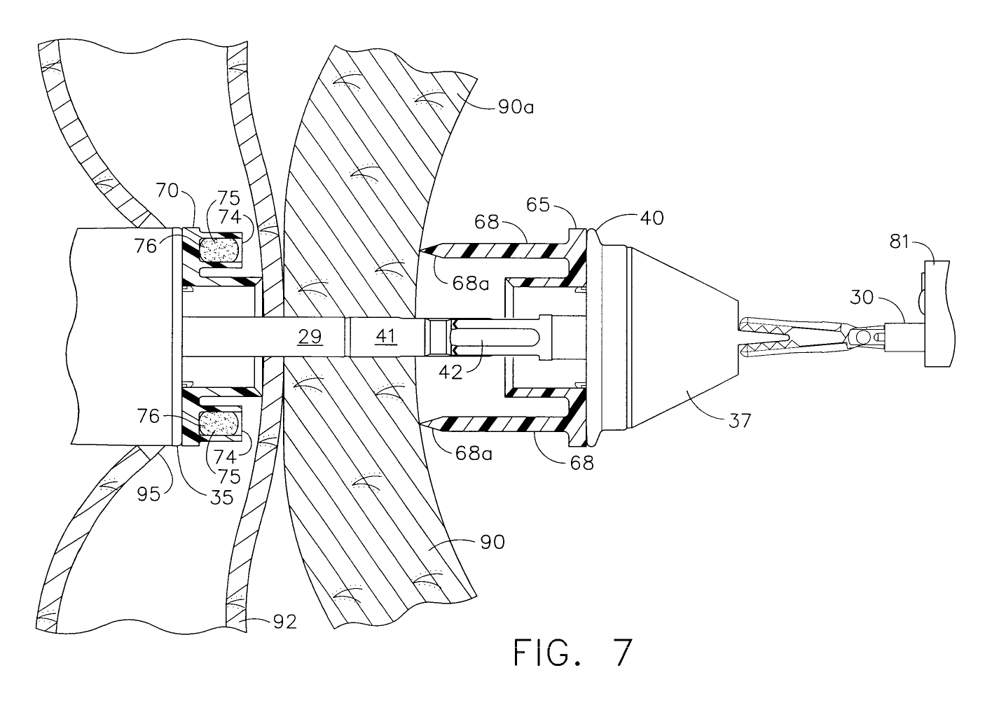

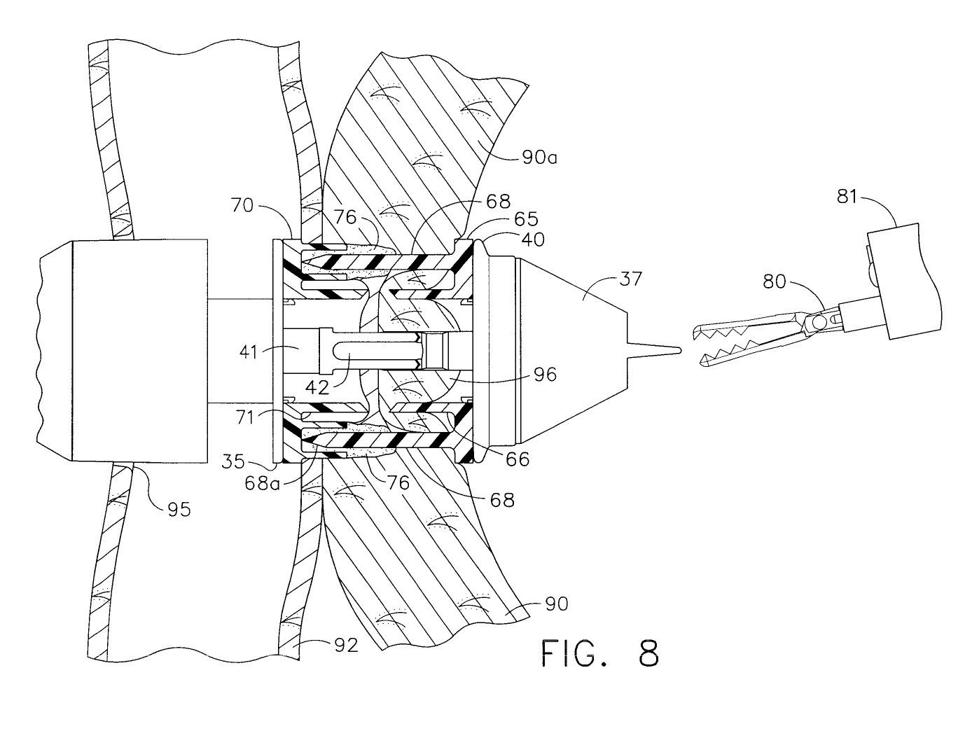

FIG. 7 shows a distal end of fastener applying instrument 25 with releasably attached second clamping ring 70 fully pushed into incision 95 in small intestine 92. This action pushes second ring 71 (FIG. 2) against an inner surface of small intestine 92 and an outer surface of small intestine 92 against stomach wall 90a. The hollow shaft 41 of distal clamp member 40 is slipped over trocar 29 protruding through the stomach wall 90a and hollow shaft 41 is rotatably aligned to trocar 29 by an alignment feature such as a flat or key (not shown). The alignment feature also aligns the first clamp ring 65 and the second clamp ring 70 by aligning points 68aand members 68 of first clamping ring 65 with sockets 74 in second clamping ring 70. Distal clamp member 40 is removably constrained onto trocar 29 by locking clamps 42. As shown, points 68aof spacers 68 extend from first clamping ring 65 and are poised to pierce stomach wall 90. This piercing occurrs when knob 27 of fastener applying instrument 25 is turned to move distal clamp member 40 and first clamping ring 65 proximally towards second clamping ring 70 to a firing position. The action of moving distal clamp member 40 towards the firing position 45 also locks distal clamp member 40 onto trocar 29 and draws points 68aof members 68 into stomach tissue 90 (not shown) and adjacent to sockets 74 of second clamping ring 70. Actuation of firing trigger 31 moves proximal clamp member 35 from the position of FIG. 7 to the fired position of FIG. 8 and fully clamps or locks the first clamp ring 65 onto the second clamp ring 70.

FIG. 8 shows the first clamp ring 65 and the second clamp ring 70 of fastener 60 after being locked together onto tissue. Proximal clamp member 35 has been moved from the firing position of FIG. 7 to the fired position of FIG. 8 to drive second clamp ring 70 distally to lock the first clamp ring 65 and the second clamp ring 70 together. This locking action moves members 68 into sockets 74, and points 68ainto ampoules 75, to release adhesive 76 and locking first clamp ring 65 and the second clamp ring 70 together. First ring 66 and second ring 71 are brought and held together to pinch stomach and intestinal tissue therebetween. The pinching action cuts off the flow of blood to the pinched tissue 96 which will die and necrose over time. Clamping pressure from first ring 66 and second ring 71 also ensure hemostasis and prevent bleeding. Adhesive 76 ensures first clamp ring 65 and second clamp ring 70 are permanently locked together to prevent separation. Grasper 80 is shown reaching towards distal clamp member 40.

FIG. 9 shows the first clamp ring 65 and the second clamp ring 70 of fastener 60 locked together onto tissue and the distal clamp member 40 being removed. Prior to this, the firing trigger 31 of fastener applying instrument 25 (not shown) has been opened to retract proximal clamp member 35 from the fired position of FIG. 7 and back to the unfired position of FIG. 8. This action has also released second clamp ring 70 from retention features 36 on fastener applying instrument 25. Knob 27 has been rotated to open distal clamp member 40, release first clamp ring 65 from retention features 36, and unlock locking clamps 42 from the trocar 29. Fastener applying instrument 25 is being removed from the incision 95 and the abdomen, and grasper 80 is grasping and withdrawing distal clamp member 40 from the surgical site and out of the patient.

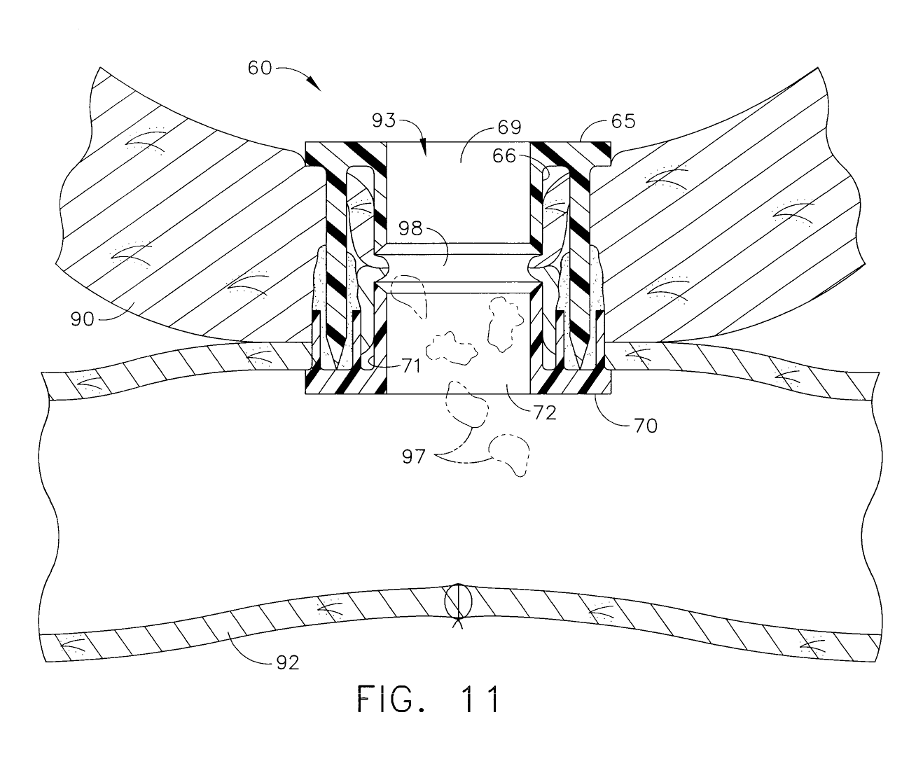

FIG. 10 shows first clamp ring 65 and the second clamp ring 70 of fastener 60 fully fastened onto the stomach 90 and small intestine 92. Pinched tissue 96 has begun to die from the cut off blood flow. Incision 95 in small intestine 92 has been closed with a plurality of sutures 97.

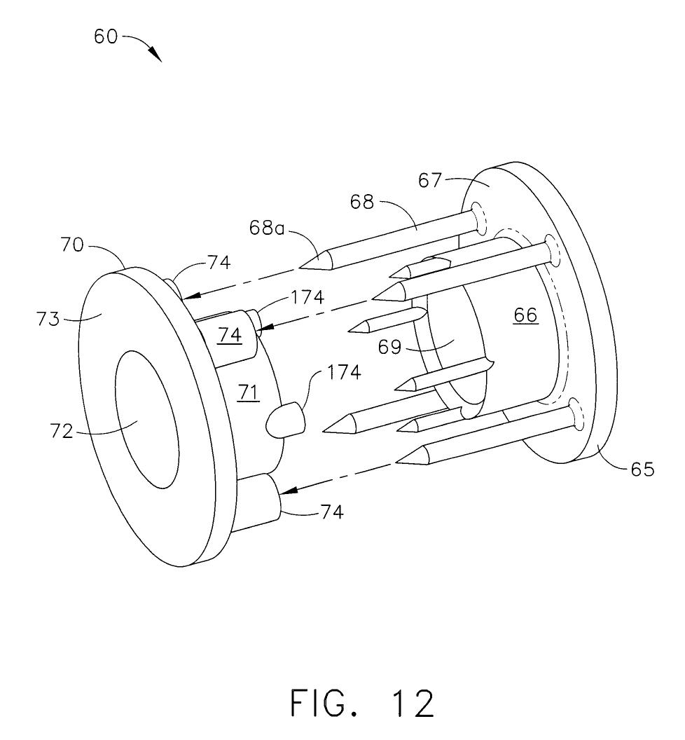

In FIG. 11, the pinched tissue 96 between first clamp ring 65 and the second clamp ring 70 has died and the necrosed tissue 97 has sloughed off and is passing into the small intestine 92 and out of the patient. A ring of healed tissue 98 has formed around the gap between first ring 66 and second ring 71. Open passageway 93 exists between stomach 90 and small intestine 92 for the passage of food therethrough and comprises first passageway 69, healed tissue 98, and second passageway 72. Fastener 60 is prevented from being dislodged or rotated by spacers 68 which pierce living tissue.

Whereas the above example method of use describes the fastener 60 being used to create an open passageway 93 between the stomach 90 and small intestine 92, the use of the fastener 60 and adhesive 76 is not limited to that particular surgery and could be adapted nor modified for use in many other surgeries. For example, the length of spacers 60 could be lengthened or shortened to make a two piece fastener with adhesive 76 that could be used to attach two pieces of tissue of a different thickness together such as intestine. Alternately, by way of example, a fastener with adhesive could be used to create an opening for the passage of feces from a patient with a colostomy and could incorporate features for the attachment of a colostomy bag.

An alternate embodiment is to make first clamping ring 65 and second clamping ring 70 from an absorbable material listed above. This would enable fastener 60 to be used as, a temporary connection to hold tissue in place until healing had occurred, after which the fastener would break down and be absorbed. If desired, during the method of attachment of the fastener 60, a sheet adhesive 76a comprising a solid film, a uniform carrier or a mesh-like carrier could be placed between tissues (not shown). Sheet adhesive 76a could be any adhesive referenced above. When first clamping ring 65 and second clamping ring 70 are clamped and locked together, the sheet adhesive 76a is actuated with moisture in the tissue or from an initiator additive 77 in an ampoule 75 in the second clamping ring 70.

Yet another alternate embodiment is a two part fastener 100 of FIG. 12 that when clamped, can use adhesive 76 to lock a plurality of spacers 160 in sockets 174, and, a first ring 166 and to a second ring 171. As shown, additional locking members 170 extend from first ring 166 and are received in sockets 175. The reception of the additional locking members 170 in sockets 175 pierce ampoules 75 containing adhesive 76 (not shown). The additional locking members 170 tie the ring structures together with adhesive 76 to better assure a complete seal of all holes and tissue contact points at the time of deployment, that only necrosis and tissue remodeling create and seal the two structures over time, and no leakage can occur. The fastener is also left in place assuring the lumen remains open, assures its structural integrity, and provides a fixation point for further trans-oral devices at a later point if necessary.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

For example, whereas the surgical implant uses pinching to necrose tissue to create an opening in the tissue, it is also within the scope of the invention to provide sharps on the first and second tissue clamping ring that can cut or partially cut a plug to create the tissue opening, or to create the opening by crushing the tissue between the first and second tissue clamping rings.

What is claimed is:

1. A surgical implant for clamping two hollow organs together and creating a passage therebetween, comprising:
a) first tissue clamping ring;
b) a second tissue clamping ring; and
c) an adhesive for locking the first and the second tissue clamping rings together in a spaced apart orientation to clamp tissue between the first and the second tissue clamping rings.

2. The surgical implant of claim 1 wherein the adhesive is at least one selected from the group consisting of a polymerizable monomer, a polymerizable 1,1,1,1-disubstituted ethylene monomer, and a cyanoacrylate formulation.

3. The surgical implant of claim 2 wherein at least one of the first and second clamping rings have at least one locking member extending therebetween, the locking member for locking the fastener in tissue and locking the first tissue clamping ring and the second tissue clamping ring member together at a spaced apart distance with the adhesive.

4. The surgical implant of claim 3 wherein the first tissue clamping ring and the second tissue clamping ring include one or more concentric rings of locking members extending between the first tissue clamping ring and the second tissue clamping ring.

5. The surgical implant of claim 4 wherein the adhesive is contained in at least one of the first and the second clamping rings.

6. The surgical implant of claim 5 wherein at least one of the first clamping rings and the second clamping rings has at least one socket therein, the socket having one or more uses selected from the group of:
a) a socket for containing the adhesive therein,
b) a socket for containing an additive therein,
c) a socket for receiving the at least one locking member therein, and
d) a socket for aligning the first clamping ring with the second clamping ring in an orientation.

7. The surgical implant of claim 6 further including at least one sealed ampoule in the socket having one or more chambers therein, wherein the contents the sealed ampoule comprises one or more selected from the group of:
a) a single part adhesive
b) a two part adhesive, and
c) an additive.

8. The surgical implant of claim 7 wherein the at least one ampoule is constructed from at least one material selected from the group of glass, ceramic, polyvinyl chloride, polyethylene, polyurethane, natural rubber, nitril rubber, butyrate rubber, polyethylene rubber, aluminum, gold, gelatin, silicone, and nylon.

9. The surgical implant of claim 7 wherein the additive is one or more additives selected from the group of anesthesia agents, sclerotic agents, necrosing agents, drugs, medicaments, adhesion initiators, micro particles, and bioactive agents,

10. The surgical implant of claim 7 wherein the at least one locking member has a piercing point, the piercing point for at least one of penetrating tissue and releasing the contents of the at least one ampoule.

11. The surgical implant of claim 7 wherein the first tissue clamping ring and the second tissue clamping ring are constructed from at least one selected from the group of stainless steel, gold, titanium, polyethylene polymers, polyetheretherketones, polylactic acid, polyglycolic acid, Polyglactin, polydioxanone, and polyglyconate.

12. A method for creating a passage in tissue between a pair of hollow organs, the method comprising;
a) providing an anastomosis device for clamping on tissue comprising
i) a first tissue clamping ring;
ii) a second tissue clamping ring;
iii) at least one locking member for spacing the first and the second locking rings apart in a spaced apart orientation, and
iv) an adhesive.
b) aligning a first hollow organ with a with a second hollow organ in a desired orientation;
c) placing one of the first tissue clamping ring and second tissue clamping rings into the first hollow organ and the other of the first tissue clamping ring and second tissue clamping rings in opposition in the second hollow organ;
d) clamping the first tissue clamping ring and the second tissue clamping ring together in a spaced apart orientation to clamp tissue therebetween and release the adhesive, the adhesive locking the first tissue clamping ring and the second tissue clamping ring together.

13. The method of claim 12 wherein after the step of clamping the first tissue clamping ring and the second tissue clamping ring together in the spaced apart orientation, including a step of necrotizing tissue clamped therebetween.

14. The method of claim 13, wherein after the step of necrotizing tissue clamped therebetween, including a step of sloughing the necrotized tissue from between the first tissue clamping ring and the second tissue clamping ring to create a passageway between the first hollow organ and the second hollow organ.

15. The method of claim 12, wherein after the step of placing one of the first tissue clamping ring and second tissue clamping rings into the first hollow organ and the other of the first tissue clamping ring and second tissue clamping rings in opposition in the second hollow organ, including a step of aligning the first tissue clamping ring and second tissue clamping ring together prior to clamping.

16. The method of claim 12 wherein during the step of providing an anastomosis device further including anastomosis clamping device having a moveable first jaw and a moveable second jaw for clamping the first tissue clamping ring and the second tissue clamping ring together in a spaced apart orientation on tissue.

17. The method of claim 12 wherein prior to the step of aligning said first tissue clamping ring and second tissue clamping ring together prior to clamping, including a step of providing a sheet adhesive and placing said sheet adhesive between the first hollow organ and the second hollow organ at the passage location.

18. The method of claim 17 wherein after the step of clamping said first tissue clamping ring and said second tissue clamping ring together in a spaced apart orientation to clamp tissue therebetween and lock said first tissue clamping ring and said second tissue clamping ring together with said adhesive, including a step of adhering the first hollow organ to the second hollow organ with said sheet adhesive.
